(19) United States
(12) Patent Application Publication
(10) Pub. No.: US 2019/0336060 A1
(43) Pub. Date: Nov. 7, 2019

(54) NON-INVASIVE MEASUREMENT SYSTEM AND METHOD USING SINGLE-SHOT SPECTRAL-DOMAIN INTERFEROMETRIC NEAR-INFRARED SPECTROSCOPY BASED ON ORTHOGONAL DISPERSION

(71) Applicant: HI LLC, Los Angeles, CA (US)

(72) Inventors: Yuecheng Shen, Guangzhou University City (CN); Haowen Ruan, Los Angeles, CA (US); Roarke Horstmeyer, Durham, NC (US); Haojiang Zhou, Los Angeles, CA (US); Jamu Alford, Simi Valley, CA (US)

(73) Assignee: HI LLC, Los Angeles, CA (US)

(21) Appl. No.: 16/392,973

(22) Filed: Apr. 24, 2019

Related U.S. Application Data
(60) Provisional application No. 62/666,949, filed on May 4, 2018.
(60) Provisional application No. 62/726,168, filed on Aug. 31, 2018.

(51) Int. Cl. A61B 5/00; G01B 11/22; G01B 9/02

(52) U.S. Cl. CPC A61B 5/4064; A61B 5/0075; G01B 9/02044; G01B 11/22; A61B 5/0082

(57) ABSTRACT

An optical measurement system comprises an optical source configured for generating source light having an optical wavelength spectrum. The optical measurement system compises an interferometer configured for splitting the source light into sample light and reference light. The interferometer is further configured for delivering the sample light into an anatomical structure, such that the sample light is scattered by the anatomical structure, resulting in physiological-encoded signal light that exits the anatomical structure. The interferometer is further configured for combining the signal light and the reference light into interference light having the optical wavelength spectrum encoded with depths of the anatomical structure. The optical measurement system further comprises a dispersive spectrometer configured for generating an optical wavelength spectrum-intensity profile from the interference light. The optical measurement system further comprises a processor configured for determining a depth of a physiological event in the anatomical structure based on the optical wavelength spectrum-intensity profile.

### RELATED APPLICATION DATA

Pursuant to 35 U.S.C. § 119(e), this application claims the benefit of U.S. Provisional Patent Application Ser. No. 62/666,949, filed May 4, 2018, and U.S. Provisional Patent Application Ser. No. 62/726,168, filed Aug. 31, 2018, which are expressly incorporated herein by reference.

### FIELD OF THE INVENTION

The present inventions relate to methods and systems for non-invasive measurements in the human body, and in particular, methods and systems related to detecting physiologically-dependent optical parameters in the human body, e.g., the brain.

### BACKGROUND OF THE INVENTION

Measuring neural activity in the brain is useful for medical diagnostics, neuromodulation therapies, neuroengineering, or brain-computer interfacing. Conventional methods for measuring neural activity in the brain include diffusive optical imaging techniques, which employ moderate amounts of near-infrared or visible light radiation, thus being comparatively safe and gentle for a biological subject in comparison to X-Ray Computed Tomography (CT) scans, positron emission tomography (PET), or other methods that use higher-energy and potentially harmful ionizing radiation. Moreover, in contrast to other known methods, such as functional magnetic resonance imaging (fMRI), these optically-based imaging methods do not require large magnets or magnetic shielding, and thus, can be scaled to wearable or portable form factors, which is especially important in applications, such as brain-computer interfacing.

However, because optical imaging techniques rely on light, which scatters many times inside brain, skull, dura, pia, and skin tissues, the light paths occurring in these techniques comprise random or “diffusive” walks, and therefore, only limited spatial resolution can be obtained by a conventional optical detector, often on the order of centimeters, with usable penetration depths being limited to a few millimeters. The reason for this limited spatial resolution is that the paths of photons striking the detector in such schemes are highly variable and difficult, and even impossible, to predict without detailed microscopic knowledge of the scattering characteristics of the brain volume of interest, which is typically unavailable in practice (i.e., in the setting of non-invasive measurements through skull for detecting neural activity in the brain for brain-computer interfacing). In summary, light scattering has presented challenges for optical detection techniques in achieving high spatial resolution deep inside tissue. Moreover, the diffusive nature of light propagation also creates challenges for measurements of fast changes in optical scattering inside tissue, since essentially all paths between source and detector are highly scattered to begin with.

One commercially available non-invasive imaging method, referred to as optical coherence tomography (OCT), is capable of acquiring images with high z-resolution (depth), but at a relatively shallow depth (1 mm-2 mm). Traditional OCT systems uses coherent light (typically light in the near-infrared spectrum) to capture sub-surface images within optical scattering media (such as biological tissue) at a micrometer-resolution. The OCT system directs an optical beam at biological tissue and collects a small portion of the light that reflects from sub-surface features of the biological tissue. Although most of the light directed at the biological tissue is not reflected, but rather, diffusively scatters and contributes to background that may obscure the image, OCT utilizes a holographic (or interferometric) technique to select, via optical path selection, the photons that directly reflect off of the sub-surface features (i.e., the ballistic backscattered photons), and reject photons that scatter multiple times in the biological tissue before detection.

In particular, in a traditional OCT system, light from a light source is split into two paths along two different arms of an interferometer: a reference arm and a sample arm. In the sample arm, sample light is backscattered through a sample medium, and in the reference arm, reference light is back-reflected by a mirror where it recombines with the backscattered sample light at a coupler. Interference light is formed by any sample light that has an optical path length that matches, within the coherence length of the optical source, the optical path length traveled by the reference light. The intensity of the backscattering sample light having that optical path length can then be detected within the interference light.

Previous commercial OCT systems acquire data in the time domain (TD-OCT), and coherence gates the backscattered light from various depths in the biological tissue by adjusting the position of the mirror to tune the optical path length of the reference, such that only sample light having the matching optical path length is selected for detection at any given time. Current commercial OCT systems acquire data in the Fourier domain (FD-OCT), and do not involve adjusting the delay of the reference arm, and thus do not coherence gate, but rather involve acquiring an interferometric signal as a function of optical wavelength by combining the sample light and the reference light from a source with a finite spectral width at a fixed reference arm delay, and then Fourier-transforming the spectral or frequency-resolved interference as a function of photon time-of-flight to obtain the various depths in the biological tissue. It has been shown that FD-OCT has a significantly greater signal-to-noise (SNR) than TD-OCT (see Michael A. Choma, et al., “Sensitivity Advantage of Swept Source and Fourier Domain Optical Coherence Tomography,” Optics Express, Vol. 11, No. 18, 8 Sep. 2003). Two distinct methods have been developed that employ the FD approach: (1) swept-source (SS-OCT), which time-encodes optical wavelengths by rapidly tuning a narrowband source through a broad optical bandwidth; and 2) spectral domain (SD-OCT), which uses a broadband light source to achieve spectral discrimination.

Regardless of the type, the depth at which a traditional OCT system images biological tissue is limited, because at greater depths the proportion of light that escapes without scattering (i.e., the ballistic light) is too small to be detected. Thus, the clinical applications of a traditional OCT system have, thus far, been limited to imaging sub-surface features, such as obtaining high-resolution ophthalmic images of the retina. As such, traditional OCT systems are is presently insufficient for measuring neural activity in the regions of the brain at deeper depths (i.e., deeper than 2 mm).

Another type of diffusive optical imaging technique, referred to as interferometric Near-Infrared Spectroscopy (iNIRS) (see Borycki, Dawid, et al., “Interferometric Near-Infrared Spectroscopy (iNIRS) for Determination of Optical and Dynamical Properties of Turbid Media,” Optics Express, Vol. 24, No. 1, Jan. 11, 2016), has been developed. While traditional OCT utilizes low-coherence interferometry to produce cross-sectional images of biological specimens with a resolution of few micrometers and an imaging depth range of 1-2 mm, the goal of iNIRS is to use high coherence interferometry to measure optical and dynamical properties of thick scattering media at a depth on the order of a few centimeters, at the cost of reduced axial resolution.

Furthermore, the systems described above have not been demonstrated to measure fast-optical signals, which refers to changes in optical scattering that occur when light propagating through active neural tissue (e.g., active brain tissue) is perturbed through a variety of mechanisms, including, but not limited to, cell swelling, cell volume change, changes in membrane potential, changes in membrane geometry, ion redistribution, birefringence changes, etc. (see Hill D. K. and Keynes, R. D., “Opacity Changes in Stimulated Nerve,” J. Physiol., Vol. 108, pp. 278-281 (1949); Foust A. J. and Rector D. M., “Optically Teasing Apart Neural Swelling and Depolarization,” Neuroscience, Vol. 145, pp. 887-899 (2007)). Because fast-optical signals are associated with neuronal activity, rather than hemodynamic responses, fast-optical signals may be used to detect brain activity with relatively high temporal resolution.

The current state of the art of iNIRS is sufficient for studying thick scattering media, it currently requires a fast-sweeping laser source to encode the spectral information in time. Such fast-sweeping laser source is relatively expensive and is subject to instability due to changes in temperature, thereby compromising its signal-to-noise ratio (SNR).

There, thus, remains a need to reduce the cost, while increasing the stability, of high coherence interferometry systems that measure optical and dynamical properties of thick scattering media.

### SUMMARY OF THE INVENTION

In accordance with one aspect of the present inventions, a non-invasive optical measurement system comprises an optical source configured for generating source light having an optical wavelength spectrum (e.g., at least 1 nm wide, and even at least 5 nm wide). The non-invasive optical measurement system further comprises an interferometer configured for splitting the source light into sample light and reference light, delivering the sample light into an anatomical structure (e.g., a brain), such that the sample light is scattered by the anatomical structure, resulting in physiological-encoded signal light that exits the anatomical structure, and combining the signal light and the reference light into interference light having the optical wavelength spectrum encoded with a plurality of depths of the anatomical structure.

The non-invasive optical measurement system further comprises a dispersive spectrometer assembly configured for generating an optical wavelength spectrum-intensity profile from the interference light. In one embodiment, the dispersive spectrometer assembly comprises a wavelength demultiplexor configured for spatially dispersing the optical wavelength spectrum of the interference light to generate spatially dispersed interference light, and an optical detector array configured for detecting the spatially dispersed interference light, and outputting the optical wavelength spectrum-intensity profile from the detected spatially dispersed interference light. The spatially dispersed interference light may be two-dimensionally spatially dispersed interference light, and the optical detector array may be a two-dimensional optical array configured for detecting the two-dimensionally spatially dispersed interference light. In such a case, the wavelength demultiplexor may comprise a collimator configured for collimating the interference light, a cylindrical lens configured for condensing the collimated interference light into line-condensed interference light, a virtually imaged phase array (VIPA) configured for spatially dispersing the optical wavelength spectrum of the line-condensed interference light along a first dimension into one-dimensionally spatially dispersed interference light, and a diffraction grating configured for spatially dispersing the optical wavelength spectrum of the one-dimensionally spatially dispersed interference light along a second dimension orthogonal to the first dimension to create the two-dimensionally spatially dispersed interference light. The wavelength demultiplexor may further comprise a spherical lens configured for focusing the two-dimensional spatially dispersed interference light onto the two-dimensional optical detector array. In one embodiment, the period of time between delivering the sample light into the anatomical structure and outputting the optical wavelength spectrum-intensity profile is equal to or less than a speckle decorrelation time of the anatomical structure, e.g., equal to or less than 100 microseconds, and preferably equal to or less than 10 microseconds.

The non-invasive optical measurement system further comprises a processor configured for determining a depth of a physiological event (e.g., indicative of neural activity, such as a fast-optical signal) in the anatomical structure based on the optical wavelength spectrum-intensity profile. In one embodiment, the processor is configured for determining the depth of the physiological event in the anatomical structure, at least partially, by comparing the optical wavelength spectrum-intensity profile to a reference optical wavelength spectrum-intensity profile. In still another embodiment, the processor configured for determining the depth of the physiological event in the anatomical structure by scaling the wavelengths of the optical wavelength spectrum-intensity profile to generate a frequency component-intensity profile, and deriving a time-of-flight (TOF)-intensity profile correlated to the depths of the anatomical structure from the frequency component-intensity profile.

In accordance with a second aspect of the present inventions, a non-invasive optical measurement method comprises generating source light having an optical wavelength spectrum (e.g., at least 1 nm wide, and even at least 5 nm wide), splitting the source light into sample light and reference light, delivering the sample light into an anatomical structure (e.g., a brain), such that the sample light is scattered by the anatomical structure, resulting in physiological-encoded signal light that exits the anatomical structure, and combining the signal light and the reference light into interference light having the optical wavelength spectrum encoded with a plurality of depths of the anatomical structure.

The non-invasive optical measurement method further comprises generating an optical wavelength spectrum-intensity profile from the interference light. In one optical measurement method, generating the optical wavelength spectrum-intensity profile comprises spatially dispersing the optical wavelength spectrum of the interference light to generate spatially dispersed interference light, detecting the spatially dispersed interference light, and generating the optical wavelength spectrum-intensity profile from the detected spatially dispersed interference light. The spatially dispersed interference light may be two-dimensionally spatially dispersed interference light. In such a case, spatially dispersing the optical wavelength spectrum of the interference light to generate the two-dimensionally spatially dispersed interference light may comprise collimating the interference light, condensing the collimated interference light into line-condensed interference light, spatially dispersing the optical wavelength spectrum of the line-condensed interference light along a first dimension into one-dimensionally spatially dispersed interference light, and spatially dispersing the optical wavelength spectrum of the one-dimensionally spatially dispersed interference light along a second dimension orthogonal to the first dimension to create the two-dimensionally spatially dispersed interference light. The non-invasive optical measurement method may further comprise focusing the two-dimensional spatially dispersed interference light. In one optical measurement method, the period of time between delivering the sample light into the anatomical structure and outputting the optical wavelength spectrum-intensity profile is equal to or less than a speckle decorrelation time of the anatomical structure, e.g., equal to or less than 100 microseconds, and preferably equal to or less than 10 microseconds.

The non-invasive optical measurement method further comprises determining a depth of a physiological event (e.g., indicative of neural activity, such as a fast-optical signal) in the anatomical structure based on the optical wavelength spectrum-intensity profile. In one optical measurement method, the depth of the physiological event in the anatomical structure is determined, at least partially, by comparing the optical wavelength spectrum-intensity profile to a reference optical wavelength spectrum-intensity profile. In another method, the depth of the physiological event in the anatomical structure is determined by scaling the wavelengths of the optical wavelength spectrum-intensity profile to generate a frequency component-intensity profile, and deriving a time-of-flight (TOF)-intensity profile correlated to the depths of the anatomical structure from the frequency component-intensity profile.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a block diagram of a non-invasive optical measurement system constructed in accordance with one embodiment of the present inventions;

FIG. 2 are a timing diagram illustrating source light having an optical wavelength spectrum generated by the non-invasive optical measurement system of FIG. 1, and a fringe pattern in interference light resulting from the combination of sample light and reference light by the optical measurement system of FIG. 1;

FIG. 3 is a plan view of a detailed embodiment of the non-invasive optical measurement system of FIG. 1;

FIG. 4 is a perspective view of an embodiment of a dispersive spectrometer assembly used in the non-invasive optical measurement system of FIG. 3;

FIG. 5 is a plan view of an optical detector array of the dispersive spectrometer assembly of FIG. 4, particularly illustrating different optical wavelengths of interference light spatially dispered onto the optical detector array;

FIG. 6A is frequency component-intensity profile detected by the non-invasive optical measurement system of FIG. 3;

FIG. 6B is a time-of-flight (TOF)-intensity profile transformed from the frequency component-intensity profile of FIG. 6A;

FIG. 7A is a timing diagram illustrating a fringe pattern of interference light corresponding to an optical path at a relatively shallow depth;

FIG. 7B is a timing diagram illustrating a fringe pattern of interference light corresponding to an optical path at a relatively deep depth;

FIG. 8A is a timing diagram illustrating exemplary oscillation frequency components of inteference light;

FIG. 8B is a timing diagram illustrating exemplary optical path length intensities corresponding to the exemplary oscillation frequency components of FIG. 8A;

FIG. 9A is a timing diagram illustrating an exemplary signal intensity-frequency profile generated by the non-invasive optical measurement system of FIG. 3;

FIG. 9B is a plan view illustrating exemplary path lengths of photons corresponding to different frequency bands of the exemplary signal intensity-frequency profile of FIG. 9A;

FIG. 10 is a plan view of physical implementation of the non-invasive optical measurement system of FIG. 1;

FIG. 11A is one profile view of one arrangement of the output port and input port of the wearable unit of FIG. 10, particularly illustrating the creation of a sample path in the head between the ports;

FIG. 11B is another profile view of the arrangement of the output port and input port of the wearable unit of FIG. 11; and

FIG. 12 is a flow diagram illustrating one method used by the optical measurement systems of FIG. 1 to non-invasively localize a physiological event within an anatomical structure.

### DETAILED DESCRIPTION OF THE EMBODIMENTS

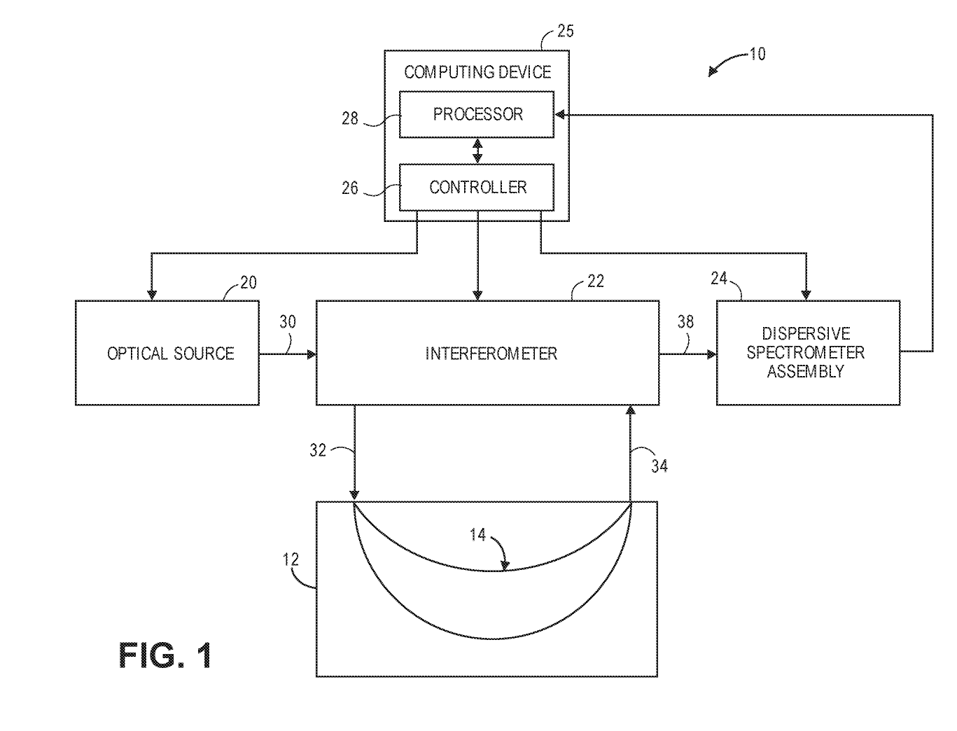

Referring now to FIG. 1, a generalized embodiment of an optical measurement system 10 constructed in accordance with the present inventions will be described. The optical measurement system 10 is a holographic optical system (i.e., a system that mixes detected signal light against reference light in order to increase the signal-to-noise ratio (SNR) of the relevant signal), and in particular interferometric Near-Infrared Spectroscopy (iNIRS) systems. As such, the optical measurement system 10 focuses on the measurement of multiple-scattered signal light of depth-correlated optical path lengths, and is capable of detecting physiological events in tissue at a penetration depth of multiple centimeters, as opposed to ballistic signal light measured by a traditional Optical Coherence Tomography (OCT) system.

However, unlike a conventional iNIRS system, which requires a fast-sweeping laser source, the non-invasive optical measurement system 10 does not require usage of an expensive tuneable frequency laser (i.e., a swept-source laser), but rather uses a relatively inexpensive laser source, and acquires a time-of-flight (TOF) profile from the spectral information of the physiological-encoded signal light in a single shot to determine the depth of a physiological event in an anatomical structure 12.

In particular, the optical measurement system 10 is designed to non-invasively acquire physiological-encoded signal light (i.e., signal light representative of a physiologically-dependent optical parameter) in the anatomical structure 12, processing the physiological-encoded signal light, and determining the presence and depth of a physiological event in the anatomical structure 12 based on the processed physiological-encoded signal light. In the illustrated embodiment, the anatomical structure 12 is a brain, in which case, the optical measurement system 10 may identify the presence and location of neural activity within the brain 12. Although for exemplary purposes, the non-invasive optical measurement system 10 is described as acquiring physiological-encoded data from brain tissue, variations of such optical measurement system 10 may be used to acquire physiological-encoded data from other anatomical structures of a human body, animal body and/or biological tissue.

In the illustrated embodiment, the physiological-encoded data acquired by the optical measurement system 10 is neural-encoded data, and the physiological event is a fast-optical signal. Fast-optical signal refers to changes in optical scattering that occur when light propagating through active neural tissue (e.g., active brain tissue) is perturbed through a variety of mechanisms, including, but not limited to, cell swelling, cell volume change, changes in membrane potential, changes in membrane geometry, ion redistribution, birefringence changes, etc. Fast-optical signals are associated with neuronal activity, rather than hemodynamic responses, and fast-optical signals may be used to detect brain activity with relatively high temporal resolution. In alternative embodiments, the physiological event may be a slower hemodynamic change, e.g., Doppler shift due to moving blood flow, changes in blood volume, metabolism variations such a blood oxygen changes. However, as will be described in further detail below, the non-invasive optical measurement system 10, when properly tuned to a specific type of physiological event, is capable of decoding light propagating through the brain to detect any physiological event that causes a change in an optical property of the neural activity in the brain 12.

The neural activity information (or the acquired neural-encoded data from which it is derived) may be transmitted to external programmable devices for use (e.g., computed, processed, stored, etc.) therein, e.g., medical devices, entertainment devices, neuromodulation stimulation devices, lie detection devices, alarm systems, educational games, brain interface devices, etc., and/or may be used internally to adjust the detection parameters of the optical measurement system 10, such as increasing or decreasing the strength of the optical source and/or data compression and/or analysis, such a Fast Fourier Transform (FFT) and/or statistical analysis.

Although the non-invasive optical measurement system 10, for purposes of brevity, is described herein as acquiring neural-encoded data from the brain 12 by using a single fixed source-detector arrangement to create one sample path 14 through the brain 12 in a single measurement period, in a practical implementation capable of three-dimensionally localizing the fast-optical signal in the brain 12, variations of the optical measurement system 10 may utilize more complex source-detector arrangements (e.g., single-source multi-detector, multi-source single-detector, or multi-source multi-detector) to simultaneously create multiple sample paths spatially separated from each other within the brain 12 in a single measurement period, or may utilize a movable source-detector arrangement to sequentially create multiple sample paths over several measurement periods, as described in U.S. Provisional Patent Application Ser. No. 62/692,074, entitled “Frequency Domain Optical Spectroscopy For Neural Decoding,” U.S. patent application Ser. No. 16/379,090, entitled “Non-Invasive Frequency Domain Optical Spectroscopy For Neural Decoding,” and U.S. Provisional Patent Application Ser. No. 62/692,124, entitled “Interferometric Frequency-Swept Source and Detector in a Photonic Integrated Circuit,” which are expressly incorporated herein by reference. Thus, the non-invasive optical detection system 10 may detect and localize physiological events associated with neural activity in the brain, including fast-optical signals, in three-dimensions, with two of the dimensions represented as an x-y plane spanning the surface of the brain 12 encoded within the spatially separated multiple sample paths and the third dimension (z-dimension or depth into the brain 12) being encoded within frequency components of photons propagating along the sample paths 14.

Referring still to FIG. 1, the optical measurement system 10 generally comprises an optical source 20, an interferometer 22, a dispersive spectrometer assembly 24, a computing device or other similar device 25, which all operate together to non-invasively detect the presence and depth of a fast-optical signal in the brain 12. The computing device 25 comprises a controller 26, a processor 28, a memory (not shown), a display (not shown), and an input device (not shown). The computing device 25 can, e.g., be a computer, tablet, mobile device, or any other suitable device for processing information. The computing device 25 can be local to the user or can include components that are non-local to the user. For example, in at least some embodiments, the user may operate a terminal that is connected to a non-local computing device. In other embodiments, the memory can be non-local to the user. The computing device 25 can utilize any suitable processor 28, including one or more hardware processors that may be local to the user or non-local to the user or other components of the computing device 25. The processor 28 is configured to execute instructions provided to the processor 28, as described below.

Any suitable memory can be used for the computing device 25. The memory can be a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

Communication methods provide another type of computer readable media; namely communication media. Communication media typically embodies computer-readable instructions, data structures, program modules, or other data in a modulated data signal. The term “modulated data signal” can include a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The display can be any suitable display device, such as a monitor, screen, or the like, and can include a printer. In some embodiments, the display is optional. In some embodiments, the display may be integrated into a single unit with the computing device 25, such as a tablet, smart phone, or smart watch. The input device can be, for example, a keyboard, mouse, touch screen, track ball, joystick, voice recognition system, or any combination thereof, or the like.

Although the controller 26 and processor 28 are described herein as being separate components, it should be appreciated that portions or all functionality of the controller 26 and processor 28 may be performed by a single component. Furthermore, although all of the functionality of the controller 26 is described herein as being performed by a single component, and likewise all of the functionality of the processor 28 is described herein as being performed by a single component, such functionality each of the controller 26 and the processor 28 may be distributed amongst several computing devices. Moreover, it should be appreciated that those skilled in the art are familiar with the terms “controller” and “processor,” and that they may be implemented in software, firmware, hardware, or any suitable combination thereof.

In this embodiment, only a single source-detector arrangement is described, although as discussed above, the optical measurement system 10 may employ a complex source-detector arrangement. The optical source 20 is configured for generating source light 30. The optical source 20 may receive power from a drive circuit (not shown), which may include control inputs for receiving control signals from the controller 24 that cause the optical source 20 to emit the source light 30 at a selected time, duration, and intensity. In the preferred embodiment, the optical source 20 may be a continuous wave (CW) optical source, although in alternative embodiments, the optical source 20 may be a pulsed wave (PW) optical source that is alternately turned on and off.

The optical source 20 may take the form of a super luminescent diode (SLD), although other light sources, e.g., a distributed feedback (DFB) laser, a light emitting diode (LED), a diode-pumped solid-state (DPSS) laser, a laser diode (LD), a super luminescent light emitting diode (sLED), a titanium sapphire laser, and/or a micro light emitting diode (mLED), or similar laser to achieve relatively broad spectral linewidths and extremely high amplitude stability, among other optical sources, may be used. The optical source 20 preferably has an optical bandwidth of several nanometers, e.g., at least 1 nm, and preferably at least 5 nm. As will be described in further detail below, the broader the optical bandwidth of the optical source 20, the higher the spatial resolution of the measurement.

The optical source 20 may have either a predefined coherence length or a variable coherence length. Since the goal of the non-invasive optical measurement system 10 is to measure optical and dynamic properties deeper in depth within brain tissue, as opposed to acquiring images of the brain tissue at a shallow depth by traditional OCT systems, the optical source 20 preferably has an spectral linewidth narrower by several orders of magnitude than in typical OCT systems, enabling the measurement of distinctly longer optical path lengths (of up to tens of centimeters) at the cost of reduced resolution (of the order of millimeters).

The source light 30 may be ultraviolet (UV) light, visible light, and/or near-infrared and infrared light, and may have any suitable wavelength, e.g., in the range of 350 nm-1800 nm. The source light 30 may be close to monochromatic in nature, comprising approximately a single-wavelength light, or the source light 30 may have multiple wavelengths (e.g., white light).

Notwithstanding the foregoing, it is preferred that the optical wavelength of the source light 30 be selected to maximize sensitivity to the specific physiological event of interest. For example, in the preferred case where the physiological event of interest is the presence of a fast-optical signal, an optical wavelength greater than 850 nm may be used for the source light 30. Optionally, an optical wavelength equal to or greater than 1000 nm may be used for the source light 30 to maximize penetration. In the additional or alternative case where the physiological event of interest is a change in the blood oxygen concentration, an optical wavelength in the range of 650 nm to 750 nm may be used for the source light 30. Multiple optical wavelengths can be used for the source light 30 to allow different physiological events to be distinguished from each other. For example, source light 30 having two optical wavelengths of 900 nm and 700 nm can be respectively used to resolve fast-optical signals and blood oxygenation. Alternatively, the wavelength of the source light 30 can be selected to maximize the detector sensitivity.

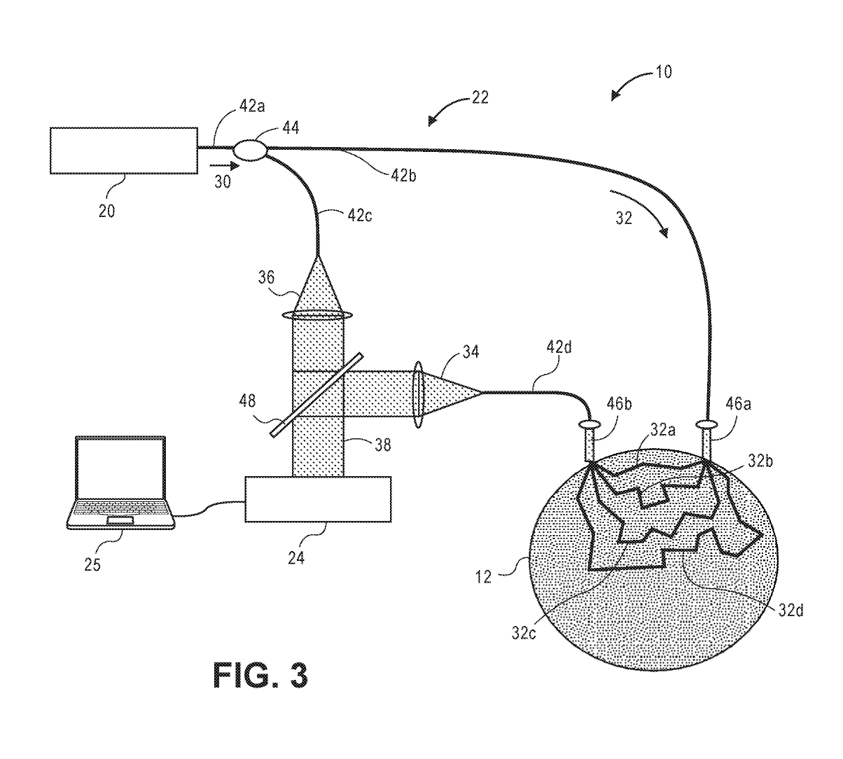

The interferometer 22 is configured for splitting the source light 30 from the optical source 20 into sample light 32, which is delivered to the brain 12 along the sample path 14 of a sample arm and exits the brain 12 as physiological-encoded (in this case, neural-encoded) signal light 34, and reference light 36 (shown in FIG. 3), which propagates along a reference arm outside of the brain 12. The interferometer 22 is further configured for combining the neural-encoded signal light 34 and the reference light 36 into interference light 38 (in this case, an interference speckle light) having an array of spatial components (in this case, speckle light grains) and a plurality of oscillation frequency components defined by the bandwidth of the source light 30.

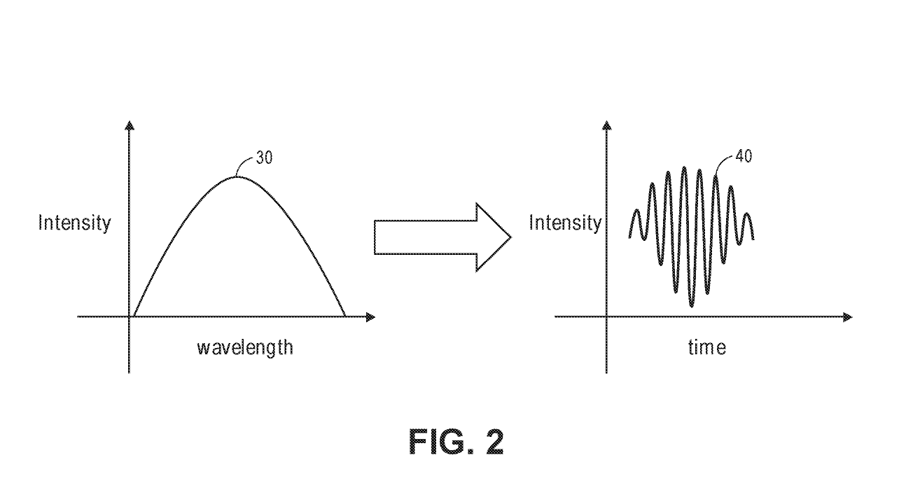

Because the neural-encoded signal light 34 comprises a collection of the scattered light of different optical path lengths comprising a range of optical wavelengths (by virtue of the source light 30 having a broad bandwidth), the interference light 38 created by the dynamic interference between the neural-encoded signal light 34 and reference light 36 results in a fringe pattern 40, as illustrated in FIG. 2. Such a fringe pattern 40 is a composite of the frequency components of the interference light 38, and it is the intensities of these frequency components that the dispersive spectrometer 24 will spatially isolate for measurement.

In particular, the dispersive spectrometer assembly 24 is configured for receiving the interference light 38 and outputting an optical wavelength spectrum-intensity profile (i.e., intensity values of the optical wavelengths of the interference light 38). As will be described in further detail below, the processor 28 is configured for determining a depth of the fast-optical signal in the brain 12 based on the optical wavelength spectrum-intensity profile output by the dispersive spectrometer assembly 24.

Referring to FIG. 3, a more detailed implementation of the optical measurement system 10 will now be described. In this implementation, the interferometer 22 is optical fiber-based (i.e., uses optical fibers to direct light between the components), although in alternative embodiments, the interferometer 22 may direct light via free-space propagation between the components using optics, such as mirrors, as further illustrated in U.S. Provisional Patent Application Ser. No. 62/637,703, entitled “Ultrasound Modulating Optical Tomography Using Reduced Laser Pulsed Duration,” U.S. patent application Ser. No. 16/266,818, entitled “Ultrasound Modulating Optical Tomography Using Reduced Laser Pulsed Duration,” U.S. Provisional Patent Application Ser. No. 62/657,634, entitled “Balanced Holography Technique for Imaging in Highly Scattering Medium,” U.S. Patent Application Ser. No. 16/299,067, entitled “Non-Invasive Optical Detection Systems and Methods in Highly Scattering Medium,” U.S. Provisional Patent Application Ser. No. 62/667,770, entitled “Ultrasound-Mediated Optical Detection,” and U.S. patent application Ser. No. 16/382,461, entitled “Non-Invasive Optical Detection System and Method,” which are expressly incorporated herein by reference.

The interferometer 22 comprises an input optical fiber 42a that optically couples the interferometer 22 to the optical source 20 for receiving the source light 30 from the optical source 20; an optical fiber-based optical beam splitter 44 for splitting the source light 30 into the sample light 32 and the reference light 36, and a sample arm optical fiber 42b and a reference arm optical fiber 42c for respectively propagating the sample light 32 and reference light 36 along the sample arm and reference arm of the interferometer 22.

The optical beam splitter 44 may not necessarily split the source light 30 equally into the sample light 32 and reference light 36, and it may actually be more beneficial for the optical beam splitter 44 to split the source light 30 unevenly, such that the intensity of the sample light 32 is less than the intensity of the reference light 36 (e.g., 99/1 power ratio), since much of the sample light 32 will be lost after passing through the brain 12. That is, the intensity of the sample light 32 should be boosted relative to the reference light 36 to compensate for the losses incurred by the sample light 32 as it passes through the brain 12 and the fact that only a small portion of neural-encoded signal light 34 (described below) exiting the brain 12 will be detected.

The sample arm optical fiber 42b delivers the sample light 32 via an output port 46a into the brain 12, such that the sample light 32 scatters diffusively through the brain 12, and back out again, exiting as the neural-encoded signal light 34. As it scatters diffusively through the brain 12, various portions of the sample light 32 will take different paths through the brain 12. For purposes of brevity, only four sample light portions 32a-32d are illustrated as traveling along optical paths of different lengths (from shallow to deeper depths), which combined into the exiting neural-encoded signal light 34, although it should be appreciated that the diffused sample light 32 will travel along many more optical paths through the brain 12. The interferometer 22 further comprises an output optical fiber 42d configured for receiving the neural-encoded signal light 34 from the brain 12 via an input port 46b.

The interferometer 22 comprises an optical beam combiner 48 configured for receiving the neural-encoded signal light 34 from the output optical fiber 42d, receiving the reference light 36 from the reference arm optical fiber 42c, and combining the neural-encoded signal light 34 and reference light 36 via superposition to generate the interference light 38, which as described above has spatial components and oscillation frequency components. In the illustrated embodiment, the optical beam combiner 48 is a free-space optical beam combiner that respectively receives the neural-encoded signal light 34 and reference light 36 on different faces of the optical beam combiner 48 and outputs the interference light 38 on another different face of the optical beam combiner 48.

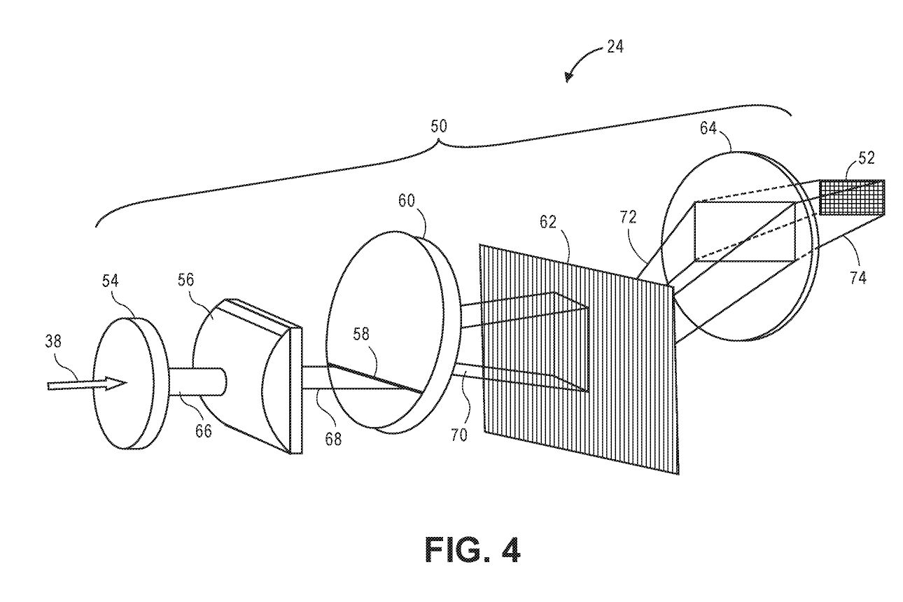

The dispersive spectrometer assembly 24 is configured for receiving the interference light 38 from the optical beam combiner 48, and outputting an optical wavelength spectrum-intensity profile. Referring to FIG. 4, the dispersive spectrometer assembly 24 comprises a wavelength demultiplexor 50 configured for spatially dispersing the optical wavelength spectrum of the interference light 38, and an optical detector array 52 (e.g., a conventional charge coupled device (CCD) camera or complementary metal-oxide-semiconductor (CMOS) camera) configured for detecting the spatially dispersed optical wavelength spectrum of the interference light 38, and outputting the optical wavelength spectrum-intensity profile. In the illustrated embodiment, the optical wavelength spectrum of the interference light 38 is spatially dispersed into two dimensions, and the optical detector array 52 is a two-dimensional optical detector array.

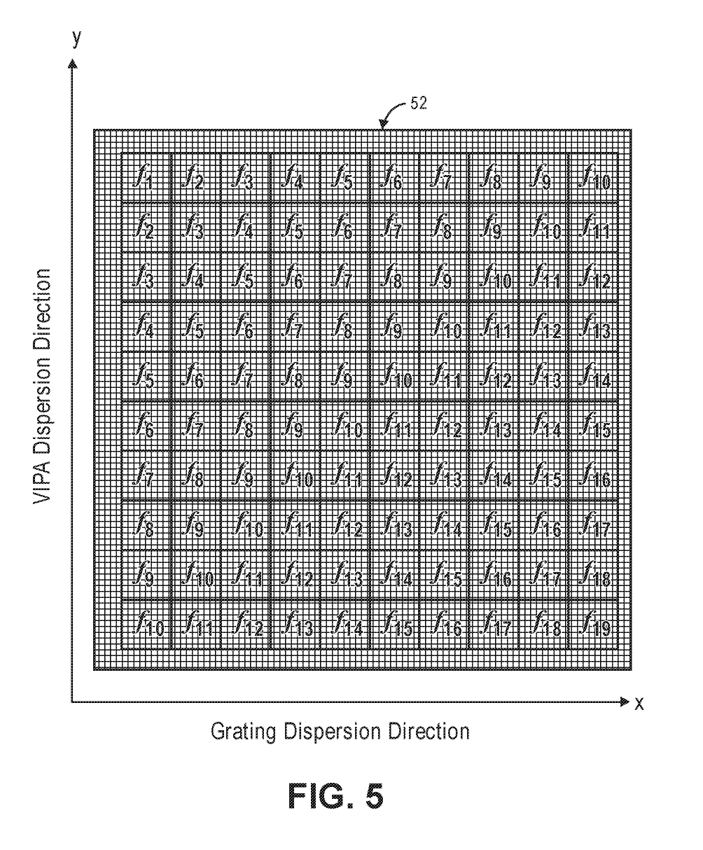

For example, as illustrated in FIG. 5, the optical wavelength spectrum of the interference light 38 is spatially dispersed in the y-direction and the x-direction, as represented by optical frequencies f₁-f₁₉. Thus, it can be appreciated that that the wavelength demultiplexor 50 encodes the one-dimensional spectral information in the interference light 38 into two-dimensional space, which can be acquired by the two-dimensional optical detector array 52 in a single shot.

Referring back to FIG. 4, the wavelength demultiplexor 50 comprises a collimator 54 configured for collimating the interference light 38, a cylindrical lens 56 configured for condensing the collimating interference light 66 into a line, and a virtually imaged phase array (VIPA) 60 having a window 58 in which the line-condensed interference light 68 enters at an oblique angle. Further details discussing VIPAs are set forth in M. Shirasaki, “Large Angular Dispersion by a Virtually Imaged Phased Array and its Applicaton to a Wavelength Demultiplexor,” Optical Letter, Vol. 21, Issue 5, pages 366-368 (1998). The VIPA 60 is configured for spatially dispersing the optical wavelength spectrum of the line-condensed interference light 68 along a first dimension (y-direction in FIG. 5) into a first spatially dispersed interference light 70. The VIPA 60 may be, e.g., a solid etalon having front and rear surfaces coated with highly reflective film, while leaving the window 58 exposed to allow entry of the line-condensed interference light. The wavelength demultiplexor 50 further comprises a high resolution diffraction grating 62 configured for spatially dispersing the optical wavelength spectrum of first spatially dispersed interference light 70 along a second dimension orthogonal to the first dimension (x-direction in FIG. 5) into two-dimension spatially dispersed interference light 72.

Thus, after passing through the wavelength demultiplexor assembly 50, the optical wavelength spectrum of the interference light 38 will be orthogonally dispersed and photons with different optical spectral information will propagate along different directions. The wavelength demultiplexor assembly 50 may further comprise a spherical lens 64 configured for focusing (i.e., mapping) the spatially dispersed optical wavelength spectrum of the interference light 72 onto the optical detector array 52. The optical detector array 52 outputs electrical signals (one for each detector or pixel) representative of the optical wavelength spectrum-intensity profile illustrated in FIG. 5.

Although only 10×10 matrix of twelve unique wavenumbers k₁-k₁₉ are illustrated in FIG. 5, it should be appreciated that the spatially dispersed optical wavelength spectrum of the interference light 38 may have many more wavenumbers. Ultimately, the number of wavenumbers will depend on the resolution of the wavelength multiplexor 50. For example, a VIPA 60 with a 2 pm spectral resolution and a diffraction grating 62 with a 100 pm spectral resolution are commercially available. Assuming a center optical wavelength μ of 800 nm, and a bandwidth Δλ. of 5 nm, the first wavelength dimension of the matrix is 50 (100 pm (resolution of the diffraction grating 62) divided by the 2 pm (dλ resolution of the VIPA 60)), and the second wavelength dimension of matrix is also 50 (5 nm (optical bandwidth Δλ) divided by 100 pm ((resolution of the diffraction grating 62)). The duration of the time-of-flight (TOF) information equals

Δt=0.44λ²/dλc=0.44*800 nm²/(2 pm*3×10⁸ m/s)=470 ps, and the time resolution equals dt=0.44λ²/(Δλc)=0.44*800 nm²/(5 nm*×10⁸ m/s)=0.19 ps.

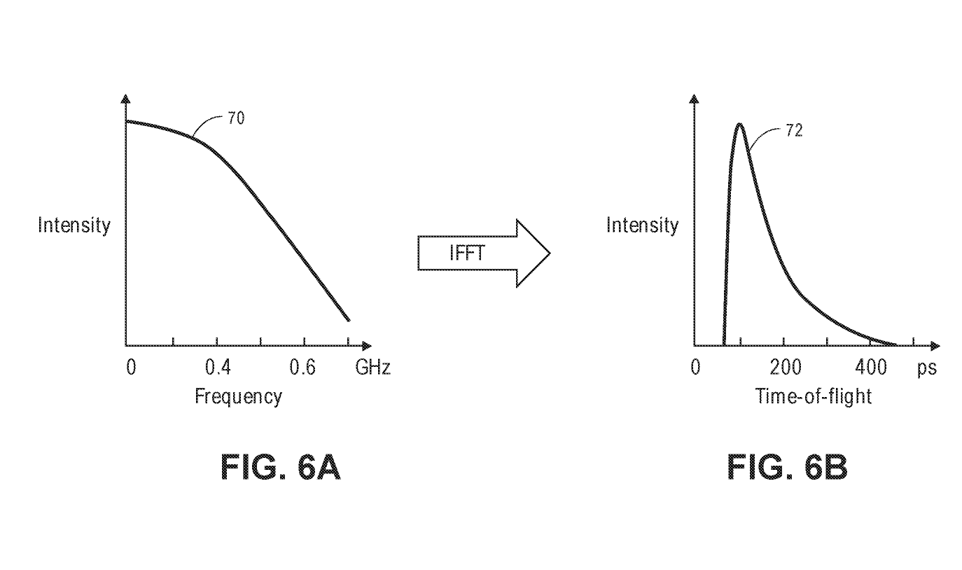

As briefly discussed above, the processor 28 is configured for determining a depth of the fast-optical signal in the brain 12 based on the optical wavelength spectrum-intensity profile output by the dispersive spectrometer assembly 24. The processor 28 may scale the wavelengths of the optical wavelength spectrum-intensity profile to generate a frequency component-intensity profile 70, as illustrated in FIG. 6A. That is, the frequencies of the frequency component-intensity profile 70 represent the differences between the center frequency of the source light 30, and the frequencies of the source light 30 across the optical bandwidth of the source light 30. Thus, the processor 28 can compute the inverse Fourier transform (e.g., using an inverse fast Fourier transform (IFFT)) of the frequency component-intensity profile 70 to obtain a time-of-flight (TOF)-intensity profile 72, as illustrated in FIG. 6B.

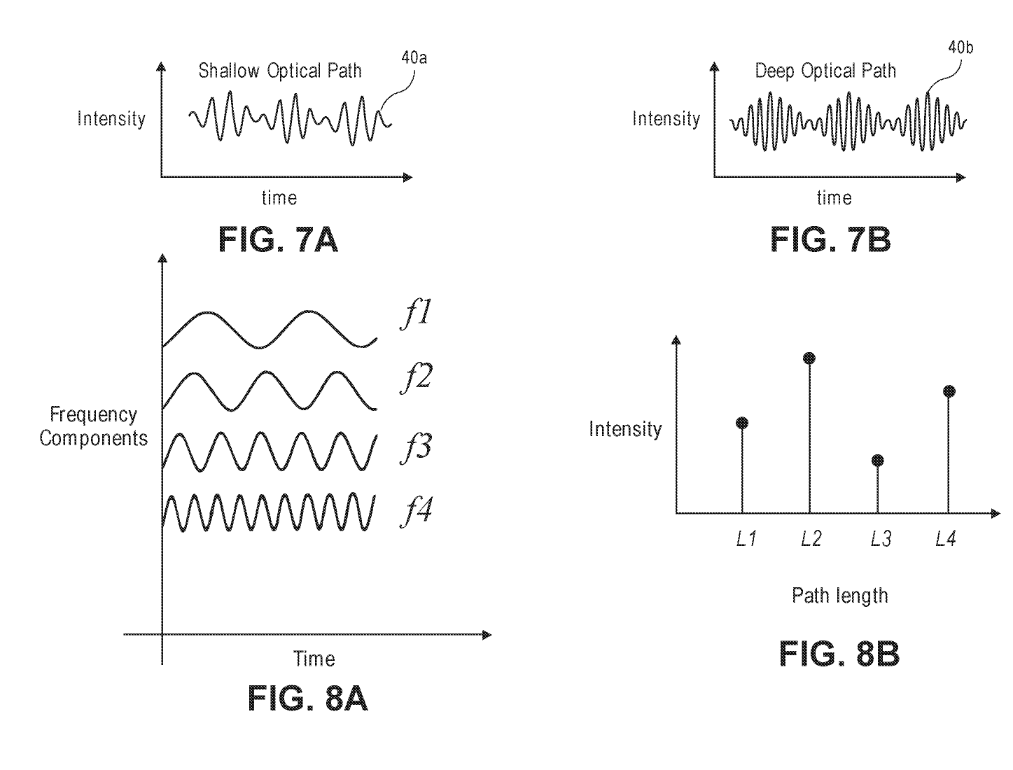

Significantly, the frequency component-intensity profile 70 comprises the intensity values of the frequency components of the fringe pattern 40 illustrated in FIG. 2, which are encoded with optical path length information, and thus, different depths of the brain 12. It follows from this that a relatively shallow depth optical path will yield a fringe pattern 40a with a relatively slow oscillation frequency (see FIG. 7A), whereas a relatively deeper depth optical path will yield a fringe pattern 40b with a relatively fast oscillation frequency (see FIG. 7B). As one example, four exemplary frequency components f1-f4 (FIG. 8A) respectively correspond to four exemplary intensities of the light at four different optical path lengths L1-L4 (FIG. 8B), which directly correlate to depths of the fast-optical signal within the brain 12.

Significantly, there is a strong correlation between the depth of penetration of photons of the sample light 32 within the brain 12 and the shape of the waveform of the detected signal light 34 in the time domain. That is, the TOF-intensity profile 72 (FIG. 6B) can be correlated to spatial depth information (i.e., the tail end of the TOF-intensity profile 72 contains relatively deeper depth information, whereas the front end of the TOF-intensity profile 72 contains relatively shallow depth information), and thus, the spatial depth of the fast-optical signal in the brain 12 may be determined. That is, it is known that the occurrence of the fast-optical signal in the brain 12 will perturb the photons of the sample light 32 at the depth of the fast-optical signal in the brain 12, thereby changing the intensity of the photons of the sample light 32 having an optical path length corresponding to that depth.

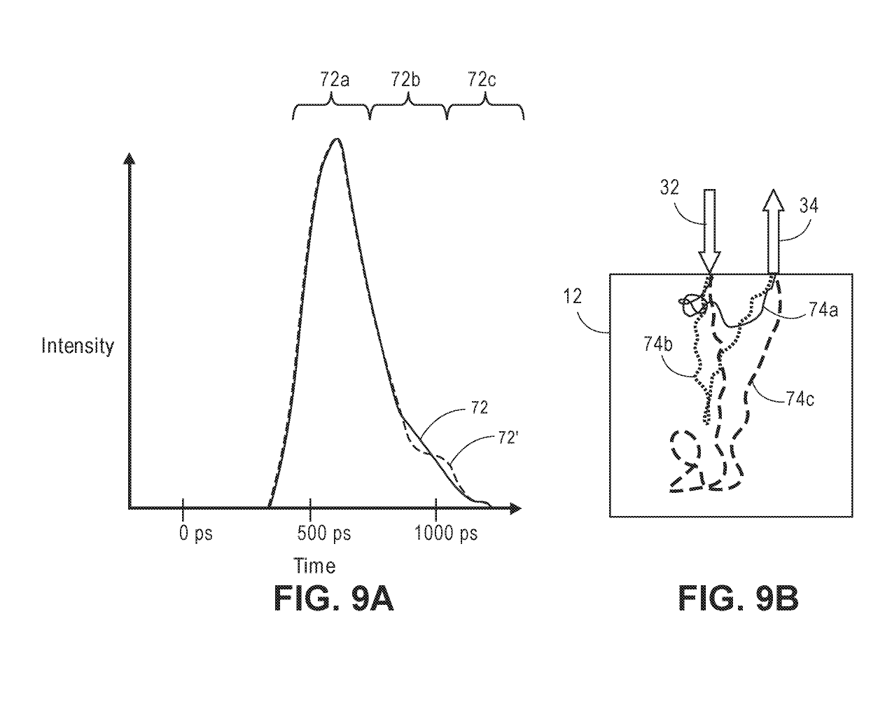

For example, as further illustrated in FIGS. 9A and 9B, a relatively early time-bin 72a of the TOF-intensity profile 72 is weighted for photons that travel a relatively short distance in the brain 12; that is, photons 74a that penetrate superficially into the brain 12; a later time-bin 72b of the TOF-intensity profile 72 is weighted for photons that travel a relatively medial distance in the brain 12; that is, photons 74b that penetrate further into the brain 12; and an even later time-bin 72c of the TOF-intensity profile 72 is weighted for photons that travel a maximum distance within the brain 12; that is, photons 74c that penetrate even further into the brain 12.

Thus, it can be appreciated that the TOF-intensity profile 72 of the detected signal light 30 contains intensity-optical path length information in which the spatial depth of a fast-optical signal is encoded, and thus, a fast-optical signal that occurs at a certain depth in the brain 12 will cause a corresponding perturbation in the TOF-intensity profile 72. For example, as shown in FIG. 9A, there exists a perturbation between the baseline TOF-intensity profile 72 in the absence of a fast-optical signal, and a TOF-intensity profile 72′ in the presence of a fast-optical signal. The fast-optical signal causes a measurable perturbation in the TOF-intensity profile 72 in time-bins 72b and 72c, indicating a change in scattering or absorption in the photons in the mid-level or maximum depth in the brain 12, and thus, the detection of a fast-optical signal at this depth in the brain 12.

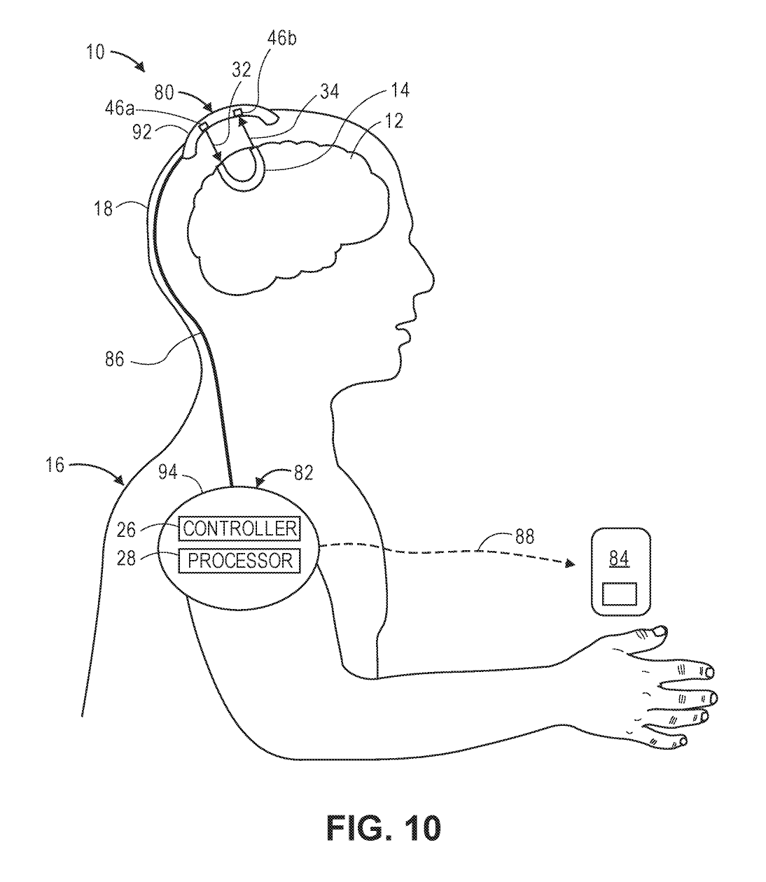

Referring now to FIG. 10, the physical implementation of the non-invasive optical measurement system 10 for use in localizing a fast-optical signal in the brain 12 of a user 16 will be described. The system 10 includes a wearable unit 80 that is configured for being applied to the user 16, and in this case, worn on the head 18 of the user 16; an auxiliary head-worn or non-head-worn unit 82 (e.g., worn on the neck, shoulders, chest, or arm) coupled to the wearable unit 80 via a wired connection 86 (e.g., electrical wires); and an optional remote processor 84 in communication with the patient-wearable auxiliary unit 82 coupled via a wired connection 88 (e.g., electrical wires). Alternatively, the non-invasive optical measurement system 10 may use a non-wired connection (e.g., wireless radio frequency (RF) signals (e.g., Bluetooth, Wifi, cellular, etc.) or optical links (e.g., fiber optic or infrared (IR)) for providing power to or communicating between the respective wearable unit 80 and the auxiliary unit 82, and/or a wired connection between the auxiliary unit 82 and the remote processor 84.

The wearable unit 80 comprises the optical source 20, and interferometer 22, dispersive spectrometer assembly 24 (illustrated in FIGS. 1 and 3), the output port 46a for emitting the sample light 32 generated by the optical source 20 (illustrated in FIGS. 1 and 3) into the head 18 of the user 16, the input port 49b configured for receiving the neural-encoded signal light 34 from the head 18 of the user 16 and delivering it to the dispersive spectrometer assembly 24 (illustrated in FIGS. 1, 3, and 4), and a support structure 92 containing the optical source 20, interferometer 22, and dispersive spectrometer assembly 24, analog portion of the processor 28, and ports 46a, 46b.

The auxiliary unit 82 comprises the controller 26 and the processor 28 (illustrated in FIGS. 1 and 3), at least a portion (e.g., any analog portion) of the processor 28 may alternatively be located in the wearable unit 80. The auxiliary unit 82 further comprises a housing 94 containing the controller 26 and processor 28. The controller 26 is configured for controlling the operational functions of the wearable unit 80, whereas the processor 28 is configured for processing the neural-encoded signal light 34 acquired by the wearable unit 80 to localize the fast-optical signal within the brain 12. The auxiliary unit 82 may additionally include a power supply (which if head-worn, may take the form of a rechargeable or non-chargeable battery), a control panel with input/output functions, a display, and memory. Alternatively, power may be provided to the auxiliary unit 82 wirelessly (e.g., by induction). The remote processor 84 may store data from previous sessions, and include a display screen.

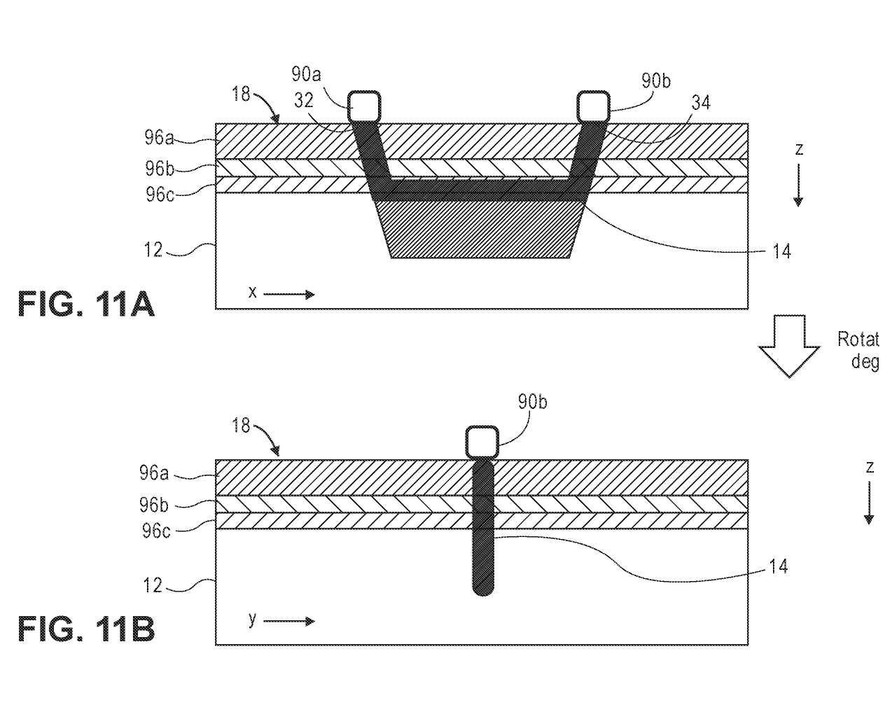

As better illustrated in FIGS. 11A and 11B, the wearable unit 80 is configured for emitting the sample light 32 into the brain 12 via the output port 46a, where is scatters, resulting in the neural-encoded signal light 34 that exits the brain 12, and is received by the input port 46b of the wearable unit 80. In particular, the sample light 32 first passes through the scalp 96a, skull 96b, and cerebral spinal fluid (CSF) 96c along a relatively straight path, enters the brain 12, then exits in reverse fashion along a relatively straight path through the CSF 96c, skull 96b, and scalp 96a, thereby defining a banana-shaped sample path 14. The wearable unit 80 may alternatively, by adding additional optical source-detector pairs, create multiple spatially separated optical paths 14 along which the light may propagate to enable x-y spatial localization of the fast-optical signal.

Referring back to FIG. 10, the support structure 92 may be shaped, e.g., have a banana, headband, cap, helmet, beanie, other hat shape, or other shape adjustable and conformable to the user's head 18, such that the ports 46a, 46b are in close contact with the outer skin of the head 18, and in this case, the scalp of the user 16. In an alternative embodiment, optical fibers (not shown) may be respectively extended from the ports 46a, 46b, thereby freeing up the requirement that the ports 46a, 46b be disposed in close proximity to the surface of the head 18. In any event, an index matching fluid may be used to reduce reflection of the light generated by the wearable unit 80 from the outer skin of the scalp. An adhesive or belt (not shown) can be used to secure the support structure 92 to the head 18 of the user 16.

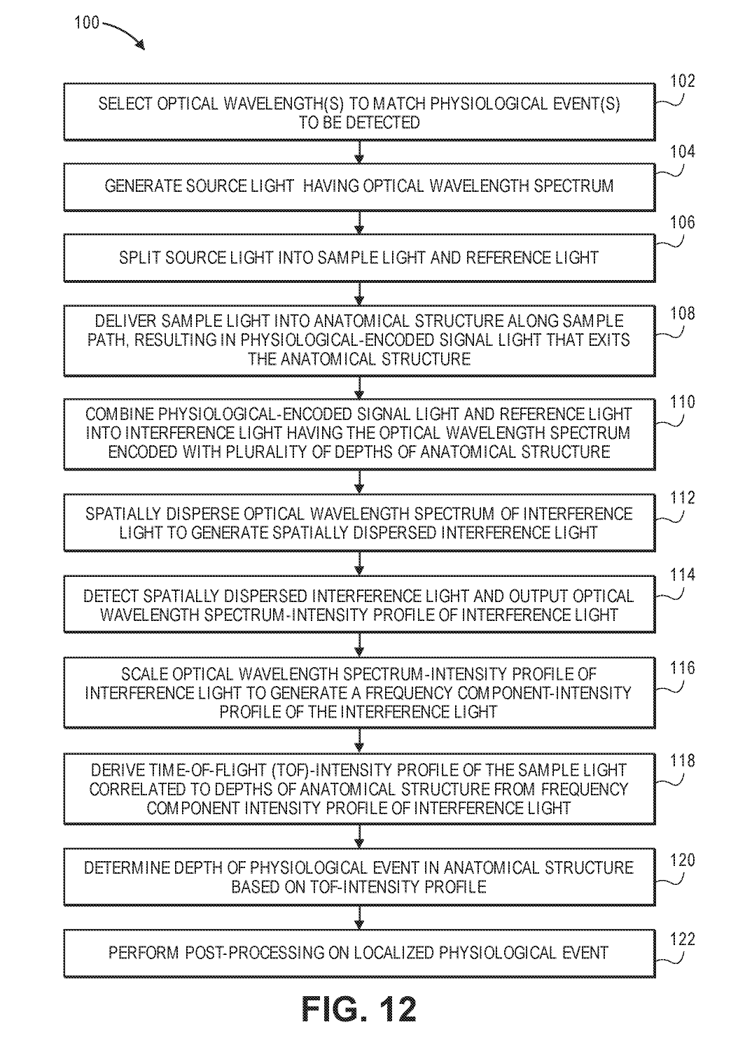

Referring to FIG. 12, having described the structure and function of the non-invasive optical measurement system 10, one particular method 100 performed by the optical measurement system 10 to non-invasively determine the depth of a physiological event (in this case, a fast-optical signal) in the anatomical structure 12 (in this case, the brain) will now be described.

First, the optical wavelength(s) of the source light 30 is selected to match the physiological event(s) to be detected in the brain 12 (step 102). In this case, the physiological event is a fast-optical signal, and thus, one optical wavelength may be greater than 850 nm. In the case where it is desirable to additionally detect blood oxygen concentration, another optical wavelength may be selected to be in the range of 650 nm to 750 nm.

Next, and with further reference to FIG. 1, the optical source 20 then generates the source light 30 with the optical wavelength spectrum during the measurement period (step 104). As discussed above, the measurement period (i.e., the period of time between delivering the sample light 32 into the brain 12 and outputting the optical wavelength spectrum-intensity profile) is equal to less than the speckle decorrelation time of the brain 12, e.g., equal to or less than 100 microseconds, and preferably, equal to or less than 10 microseconds.

The interferometer 22 (e.g., via the optical beam splitter 44) splits the source light 30 into sample light 32 and reference light 36 (step 106), delivers the sample light 32 into the brain 12 along a single sample path 14, such that the sample light 32 is scattered by the brain 12, resulting in physiological-encoded signal light 34 that exits the brain 12 (step 108), and combines, the physiological-encoded signal light 34 and the reference light 36 into interference light 38 having an optical wavelength spectrum encoded with a plurality of depths of the brain 12 (step 110).

Next, and with further reference to FIG. 4, the wavelength demultiplexor 50 of the dispersive spectrometer assembly 24 spatially disperses the optical wavelength spectrum of the interference light 38 to generate spatially dispersed interference light, and in particular, two-dimensionally spatially dispersed interference light 74 (step 112). In one method, the collimator 54 of the wavelength demultiplexor 50 collimates the interference light 38 into collimated interference light 66, the cylindrical lens 56 of the wavelength demultiplexor 50 of the dispersive spectrometer assembly 24 condenses the collimated interference light 66 into line-condensed interference light 68, the VIPA 60 of the wavelength demultiplexor 50 spatially disperses the optical wavelength spectrum of the line-condensed interference light 68 along a first dimension into one-dimensionally spatially dispersed interference light 70 (y-direction in FIG. 5), the diffractive grating 62 of the wavelength demultiplexor 50 spatially disperses the optical wavelength spectrum of the one-dimensionally spatially dispersed interference light 70 along a second dimension orthogonal to the first dimension (x-direction in FIG. 5) to create the two-dimensionally spatially dispersed interference light 72, and the spherical lens 64 of the wavelength demultiplexor 50 focuses the two-dimensional spatially dispersed interference light 72 to create a focused two-dimensionally spatially dispersed interference light 74.

Next, the optical detector array 52 of the dispersive spectrometer assembly 24 detects the focused two-dimensionally spatially dispersed interference light 74, and outputs the optical wavelength spectrum-intensity profile of interference light 38 (step 114). The processor 28 then determines a depth of a fast-optical signal in the brain 12 based on the optical wavelength spectrum-intensity profile of the interference light 38. In particular, the processor 28 scales the optical wavelength spectrum-intensity profile to generate a frequency component-intensity profile 70 of the interference light 38 (see FIG. 6A) (step 116), and then derives a time-of-flight (TOF)-intensity profile 72 of the sample light 32 correlated to the depths of the brain 12 from the frequency component-intensity profile 70 (see FIG. 6B), e.g., by using an inverse Fourier transform (e.g., an inverse fast Fourier transform (IFFT)) (step 118). The processor 28 then determines the depth of the fast-optical signal in the brain 12 based on the TOF-intensity profile 72 (step 120), e.g., in the manner described above with respect to FIGS. 9A and 9B.

In the case where multiple sample paths 14 through the brain 12 are created using complex source-detector arrangements (e.g., single-source multi-detector, multi-source single-detector, or multi-source multi-detector) to simultaneously create multiple sample paths 14 spatially separated from each other within the brain 12 in a single measurement period, or by using a movable source-detector arrangement, the processor 28 may also localize the fast-optical signal in an x-y plane along the surface of the brain 12, such that a three-dimensional location of the fast-optical signal within the brain 12 is determined. The processor 28 may then perform post-processing on the localized fast-optical signal, e.g., determining the level and location of neural activity within the brain 12 (step 122).

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A non-invasive optical measurement system, comprising:
an optical source configured for generating source light having an optical wavelength spectrum;
an interferometer configured for splitting the source light into sample light and reference light, delivering the sample light into an anatomical structure, such that the sample light is scattered by the anatomical structure, resulting in physiological-encoded signal light that exits the anatomical structure, and combining the signal light and the reference light into interference light having the optical wavelength spectrum encoded with a plurality of depths of the anatomical structure;
a dispersive spectrometer assembly configured for generating an optical wavelength spectrum-intensity profile from the interference light; and
a processor configured for determining a depth of a physiological event in the anatomical structure based on the optical wavelength spectrum-intensity profile.

2. The non-invasive optical measurement system of claim 1, wherein the dispersive spectrometer assembly comprises:
a wavelength demultiplexor configured for spatially dispersing the optical wavelength spectrum of the interference light to generate spatially dispersed interference light; and
an optical detector array configured for detecting the spatially dispersed interference light, and outputting the optical wavelength spectrum-intensity profile from the detected spatially dispersed interference light.

3. The non-invasive optical measurement system of claim 2, wherein the spatially dispersed interference light is two-dimensionally spatially dispersed interference light, and the optical detector array is a two-dimensional optical array configured for detecting the two-dimensionally spatially dispersed interference light.

4. The non-invasive optical measurement system of claim 3, wherein the wavelength demultiplexor comprises:
a collimator configured for collimating the interference light;
a cylindrical lens configured for condensing the collimated interference light into line-condensed interference light;
a virtually imaged phase array (VIPA) configured for spatially dispersing the optical wavelength spectrum of the line-condensed interference light along a first dimension into one-dimensionally spatially dispersed interference light; and
a diffraction grating configured for spatially dispersing the optical wavelength spectrum of the one-dimensionally spatially dispersed interference light along a second dimension orthogonal to the first dimension to create the two-dimensionally spatially dispersed interference light.

5. The non-invasive optical measurement system of claim 4, wherein the wavelength demultiplexor further comprises a spherical lens configured for focusing the two-dimensional spatially dispersed interference light onto the two-dimensional optical detector array.

6. The non-invasive optical measurement system of claim 1, wherein the anatomical structure is a brain.

7. The non-invasive optical measurement system of claim 6, wherein the physiological event is indicative of neural activity.

8. The non-invasive optical measurement system of claim 7, wherein the physiological event is a fast-optical signal.

9. The non-invasive optical measurement system of claim 1, wherein a period of time between delivering the sample light into the anatomical structure and outputting the optical wavelength spectrum-intensity profile is equal to or less than a speckle decorrelation time of the anatomical structure.

10. The non-invasive optical measurement system of claim 1, wherein a period of time between delivering the sample light into the anatomical structure and outputting the optical wavelength spectrum-intensity profile is equal to or less than 100 microseconds.

11. The non-invasive optical measurement system of claim 1, wherein a period of time between delivering the sample light into the anatomical structure and outputting the optical wavelength spectrum-intensity profile is equal to or less than 10 microseconds.

12. The non-invasive optical measurement system of claim 1, wherein the processor is configured for determining the depth of the physiological event in the anatomical structure, at least partially, by comparing the optical wavelength spectrum-intensity profile to a reference optical wavelength spectrum-intensity profile.

13. The non-invasive optical measurement system of claim 1, wherein the optical wavelength spectrum is at least 1 nm wide.

14. The non-invasive optical measurement system of claim 1, wherein the optical wavelength spectrum is at least 5 nm wide.

15. The non-invasive optical measurement system of claim 1, wherein the processor is configured for determining the depth of the physiological event in the anatomical structure by scaling the wavelengths of the optical wavelength spectrum-intensity profile to generate a frequency component-intensity profile, and deriving a time-of-flight (TOF)-intensity profile correlated to the depths of the anatomical structure from the frequency component-intensity profile.

16. A non-invasive optical measurement method, comprising:
generating source light having an optical wavelength spectrum;
splitting the source light into sample light and reference light;
delivering the sample light into an anatomical structure, such that the sample light is scattered by the anatomical structure, resulting in physiological-encoded signal light that exits the anatomical structure;
combining the signal light and the reference light into interference light having the optical wavelength spectrum encoded with a plurality of depths of the anatomical structure;
generating an optical wavelength spectrum-intensity profile from the interference light; and
determining a depth of a physiological event in the anatomical structure based on the optical wavelength spectrum-intensity profile.

17. The non-invasive optical measurement method of claim 16, wherein generating the optical wavelength spectrum-intensity profile comprises:
spatially dispersing the optical wavelength spectrum of the interference light to generate spatially dispersed interference light;
detecting the spatially dispersed interference light; and
generating the optical wavelength spectrum-intensity profile from the detected spatially dispersed interference light.

18. The non-invasive optical measurement method of claim 17, wherein the spatially dispersed interference light is two-dimensionally spatially dispersed interference light.

19. The non-invasive optical measurement method of claim 18, wherein spatially dispersing the optical wavelength spectrum of the interference light to generate the two-dimensionally spatially dispersed interference light comprises:
collimating the interference light;
condensing the collimated interference light into line-condensed interference light;
spatially dispersing the optical wavelength spectrum of the line-condensed interference light along a first dimension into one-dimensionally spatially dispersed interference light; and
spatially dispersing the optical wavelength spectrum of the one-dimensionally spatially dispersed interference light along a second dimension orthogonal to the first dimension to create the two-dimensionally spatially dispersed interference light.

20. The non-invasive optical measurement method of claim 19, further comprising focusing the two-dimensional spatially dispersed interference light.

21. The non-invasive optical measurement method of claim 19, wherein the anatomical structure is a brain.

22. The non-invasive optical measurement method of claim 21, wherein the physiological event is indicative of neural activity.

23. The non-invasive optical measurement method of claim 22, wherein the physiological event is a fast-optical signal.

24. The non-invasive optical measurement method of claim 16, wherein a period of time between delivering the sample light into the anatomical structure and outputting the optical wavelength spectrum-intensity profile is equal to or less than a speckle decorrelation time of the anatomical structure.

25. The non-invasive optical measurement method of claim 16, wherein a period of time between delivering the sample light into the anatomical structure and outputting the optical wavelength spectrum-intensity profile is equal to or less than 100 microseconds.

26. The non-invasive optical measurement method of claim 16, wherein a period of time between delivering the sample light into the anatomical structure and outputting the optical wavelength spectrum-intensity profile is equal to or less than 10 microseconds.

27. The non-invasive optical measurement method of claim 16, wherein the depth of the physiological event in the anatomical structure is determined, at least partially, by comparing the optical wavelength spectrum-intensity profile to a reference optical wavelength spectrum-intensity profile.

28. The non-invasive optical measurement method of claim 16, wherein the optical wavelength spectrum is at least 1 nm wide.

29. The non-invasive optical measurement method of claim 16, wherein the optical wavelength spectrum is at least 5 nm wide.

30. The non-invasive optical measurement method of claim 16, wherein the depth of the physiological event in the anatomical structure is determined by scaling the wavelengths of the optical wavelength spectrum-intensity profile to generate a frequency component-intensity profile, and deriving a time-of-flight (TOF)-intensity profile correlated to the depths of the anatomical structure from the frequency component-intensity profile.
